(19) United States
(12) Patent Application Publication
(10) Pub. No.: US 2005/0267076 A1
(43) Pub. Date: Dec. 1, 2005

(54) Method for improving cognitive function

(75) Inventor: Michela Gallagher, Baltimore, MD (US)

Correspondence Address: FOLEY HOAG, LLP;PATENT GROUP, WORLD TRADE CENTER WEST, 155 SEAPORT BLVD, BOSTON, MA, 02110, US

(73) Assignee: Johns Hopkins University, Baltimore, MD (US)

(21) Appl. No.: 11/129,026

(22) Filed: May 13, 2005

Related U.S. Application Data
(60) Provisional application No. 60/571,330, filed on May 14, 2004.

(51) Int. Cl. A61K 31/66; A61K 31/445

(52) U.S. Cl. 514/114; 514/319

(57) ABSTRACT

Methods and compositions for improving cognitive function by co-administration of donepezil and a GABA_{B} receptor antagonist are provided.

### CROSS-REFERENCE TO RELATED PATENT APPLICATIONS

This application claims benefit under 35 U.S.C. 119 to provisional application No. 60/571,330, filed 14 May 2004, the entire contents of which are incorporated herein by reference.

### FIELD OF INVENTION

The invention relates to methods and compositions for improving cognitive function by administering donepezil, an acetylcholinesterase inhibitor, in combination with a GABA_{B} receptor antagonist such as 3-aminopropyl-(n-butyl)-phosphinic acid.

### BACKGROUND OF THE INVENTION

Cognitive and/or degenerative brain disorders are characterized clinically by progressive loss of memory, cognition, reasoning, judgment and emotional stability, gradually leading to profound mental deterioration. Among these diseases, Alzheimer's Disease is common and is believed to represent the fourth most common medical cause of death in the United States. In 2005, Alzheimer's Disease was estimated to affect more than 4 million people in the United States, a number expected to increase within the next 50 years. Additionally, the number of patients falling in the categories of Mild Cognitive Impairment, Age-Associated Memory Impairment, Age-Related Cognitive Decline or similar diagnostic categories is also staggering. For example, according to the estimates of Barker et al. (1995) there are more than 16 million people with Age Associated Memory Impairment in the U.S. alone.

Donepezil, sold in the United States under the trade name ARICEPT®, is an acetylcholinesterase inhibitor used to treat mild to moderate dementia of the Alzheimer's type. With donepezil, Alzheimer's Disease patients show slight cognitive improvements (Barner and Gray, 1998; Rogers and Friedhoff, 1998) but the usefulness of donepezil is limited by its moderate efficacy and side effects. There is therefore still a need for effective treatment for disorders involving cognitive dysfunction.

### SUMMARY OF THE INVENTION

The present invention provides methods and compositions for improving cognitive function and in particular, improving cognitive function in subjects suffering from a disorder involving cognitive dysfunction.

In one aspect, the invention provides a method for improving cognitive function in a mammal, such as a human, by administering donepezil in combination with a GABA_{B} receptor antagonist. In an aspect, the invention provides a method for improving cognitive function in a mammal by administering donepezil in combination with ABPA (3-aminopropyl-(n-butyl)-phosphinic acid). The drugs may be administered simultaneously, optionally as a single coformulation, or at different times. When administered in combination, ABPA and donepezil have been discovered to act synergistically. Surprisingly, the combination provides benefit even when the amount of each drug administered is an amount that is suboptimal (if administered individually). Surprisingly, the combination provides benefit even when the amount of each drug administered is an amount that, if administered individually, would have little or essentially no therapeutic effect.

In another aspect, the present invention provides pharmaceutical compositions comprising a GABA_{B} receptor antagonist, such as ABPA, and donepezil. In some embodiments, the composition is provided as a unit dosage form.

### DESCRIPTION OF THE FIGURES

FIG. 1 shows the performance of rats on a retention test on a 12-arm maze following administration of ABPA, donepezil hydrochloride, or both ABPA and donepezil.

FIG. 2 shows the inter-trial interval determined for untreated rats in the object recognition task.

FIG. 3 shows dose-effect curves for ABPA and donepezil in the object recognition task.

FIGS. 4A, 4B and 4C show the effects of administering donepezil, ABPA or both ABPA and donepezil on performance in the object recognition task.

FIG. 5 shows isobolograms for the combination of donepezil and ABPA in the object recognition task, each using different “effect” levels. The dot represents the 1 mg/kg donepezil and 3 mg/kg ABPA dose combination.

### DETAILED DESCRIPTION OF THE INVENTION

In one aspect, the present invention provides methods of improving cognitive function in a subject by administering a GABA_{B} receptor antagonist in combination with donepezil. It has been discovered that when donepezil and a GABA_{B} receptor antagonist are administered in combination they have a synergistic effect and provide therapeutic effect even when administered at doses that are suboptimal or subtherapeutic when administered individually. This discovery provides several important therapeutic benefits, including: (1) a better therapeutic result can be achieved using the combination than from either component administered alone; (2) when used in the combination, donepezil can be administered at lower doses without diminishing therapeutic efficacy, thereby avoiding the side-effect profile characteristic of higher doses; and (3) when used in the combination, the GABA_{B} receptor antagonist can be administered at lower doses, resulting in reduced cost and increased convenience. An exemplary GABA_{B} receptor antagonist for use in accord with the invention is 3-aminopropyl-(n-butyl)-phosphinic acid (ABPA). The GABA_{B} receptor antagonist and donepezil can be administered simultaneously, sequentially, or in the same course of therapy, and they may be administered as co-formulations or as separate compositions. In a related aspect, the invention provides unit dosage forms and other pharmaceutical compositions for administration to improve cognition.

Improving Cognitive Function and Treating Cognitive Impairment

The methods and compositions of the invention are useful for improving cognitive function in a mammal (e.g., human, nonhuman primate, or rat). Improving cognitive function includes “promoting” cognitive function (affecting impaired cognitive function in the subject so that it more closely resembles the function of an aged-matched normal, unimpaired subject, including affecting states in which cognitive function is reduced compared to a normal subject) and “preserving” cognitive function (affecting normal or impaired cognitive function such that it does not decline or does not fall below that observed in the subject upon first presentation or diagnosis, e.g., to the extent of expected decline in the absence of treatment).

In one embodiment of the invention, the mammal has normal cognitive function which is improved. In one embodiment the mammal exhibits cognitive impairment associated with aging. In one embodiment the mammal is a human with cognitive impairment associated with a disease or disorder. In one embodiment the mammal is a human exhibiting cognitive function impairment associated with a disorder such as Alzheimer's Disease, mild cognitive impairment (MCI), age-related cognitive decline, vascular dementia, Parkinson's Disease, memory impairment associated with depression or anxiety, psychosis, Down's Syndrome, stroke, traumatic brain injury, Huntington's disease, AIDS associated dementia, schizophrenia, and attention deficit disorders. In one embodiment, the impairment of cognitive function is caused by, or attributed to, Alzheimer's disease. In another embodiment, the impairment of cognitive function is caused by, or attributed to, mild cognitive impairment (MCI). Methods for diagnosis or assessment of a subject having cognitive function impairment or a related condition are well-known in the art, and can be conducted by a physician or other medical professional. Thus, in one aspect the invention provides a method involving administering (as broadly defined herein) donepezil and a GABA_{B} receptor antagonist in combination to a subject diagnosed as exhibiting cognitive impairment, optionally due to a condition listed above.

As used herein, “treating” a condition or patient refers to taking steps to obtain beneficial or desired results, including clinical results. Beneficial or desired clinical results include, but are not limited to, alleviation or amelioration of one or more symptoms associated with disorders involving cognitive dysfunction, diminishment of extent of disease, delay or slowing of disease progression, amelioration, palliation or stabilization of the disease state, and other beneficial results, such as improvement of cognitive function or a reduced rate of decline of cognitive function.

Cognitive function can be assessed by methods known in the art, for example, a variety of tests known to those skilled in the art can be used to demonstrate cognitive impairment, or the lack thereof, in a human. These tests include, but are not limited to, the Alzheimer's Disease Assessment Scale-cognitive subscale (ADAS-cog), the clinical global impression of change scale (CIBIC-plus scale), the Alzheimer's Disease Cooperative Study Activities of Daily Living Scale (ADCS-ADL), the Mini Mental State Exam (MMSE); the Neuropsychiatric Inventory (NPI), the Clinical Dementia Rating Scale (CDR), the Cambridge Neuropsychological Test Automated Battery (CANTAB), and the Sandoz Clinical Assessment-Geriatric (SCAG). In addition, cognitive function may be measured using imaging techniques such as Positron Emission Tomography (PET), functional magnetic resonance imaging (fMRI), or Single Photon Emission Computed Tomography (SPECT) to measure brain activity. In animal model systems, cognitive impairment can be measured in any number of ways known in the art, including using the Morris Water Maze or Object Recognition Task (see examples).

As used herein, a “therapeutically effective amount” of a drug is an amount of a drug that, when administered to a subject will have the intended therapeutic effect, e.g. improving cognitive function in a subject. The full therapeutic effect does not necessarily occur by administration of one dose, and may occur only after administration of a series of doses. Thus, a therapeutically effective amount may be administered in one or more administrations.

It is also contemplated that the combination of the invention will be administered prophylactically. For example, donepezil and a GABA_{B} receptor antagonist can be administered to a subject at risk for developing a cognitive disorder.

Donepezil

Donepezil ((±)-2,3-dihydro-5,6-dimethoxy-2-[[1-(phenylmethyl)-4-piperidinyl]methyl]-1H-inden-1-one; also known as “E220”) is usually administered as the hydrochloride salt. Donepezil hydrochloride ((±)-2,3-dihydro-5,6-dimethoxy-2-[[1-(phenylmethyl)-4-piperidinyl]methyl]-1H-inden-1-one hydrochloride) is marketed in the United States as ARICEPT®. See U.S. Pat. Nos. 4,895,841, 5,985,864; 6,140,321, 6,245,911, 6,372,760. In addition to donepezil hydrochloride, other forms of donepezil, including without limitation salts, hydrates, co-crystals, enantiomers, and prodrugs can be administered in accordance with the methods of the invention.

When administered to humans, donepezil showed a cholinergic side effect profile, and the dosage administered to patients is limited by such side effects. Donepezil hydrochloride is typically administered orally at a dose of 5 or 10 mg once daily for treatment of the symptoms of mild-to-moderate Alzheimer's disease.

GABA_{B} Receptor Antagonists

As used herein, “GABA_{B} receptor antagonist” has its ordinary meaning, and refers to an agent that blocks, suppresses, or reduces GABA_{B} receptor activity. GABA_{B} receptors are localized both pre- and postsynaptically. Presynaptically GABA_{B} receptors act as inhibitory autoreceptors that upon activation reduce the release of neurotransmitters including acetylcholine, glutamate, serotonin, norepinephrine, neuropeptides, and GABA (Misgeld et al., 1995; Ong and Kerr, 2000). GABA_{B} receptor antagonists may block presynaptic GABA_{B} autoreceptor function and thus increase neurotransmitter release. GABA_{B} receptor antagonists may also antagonize GABA_{B} receptor-mediated hyperpolarization postsynaptically (Kuriyama et al., 2000), facilitate postsynaptic N-methyl-D-asparate receptor (NMDA-R) function (Pittaluga et al., 2001) and stimulate neurotrophin release (Heese et al., 2000 and U.S. Pat. App. 20020013257). For a review on GABA_{B} receptor antagonists and their therapeutic applications, see for example Bittiger et al., 1993.

An exemplary GABA_{B} receptor antagonist is 3-aminopropyl-(n-butyl)-phosphinic acid called “ABPA” (also known as “SGS742” and “CGP36742”), or a salt, prodrug, analog or derivative thereof. ABPA is a phosphoaminoacid derivative that is highly water-soluble and readily crosses the blood brain barrier. ABPA and salts thereof are described in U.S. Pat. Nos. 5,300,679 and 5,064,819; Gleiter et al., 1996; Mondadori et al., 1993; Mondadori et al., 1996; Pittaluga et al., 1997; and Steulet et al., 1996.

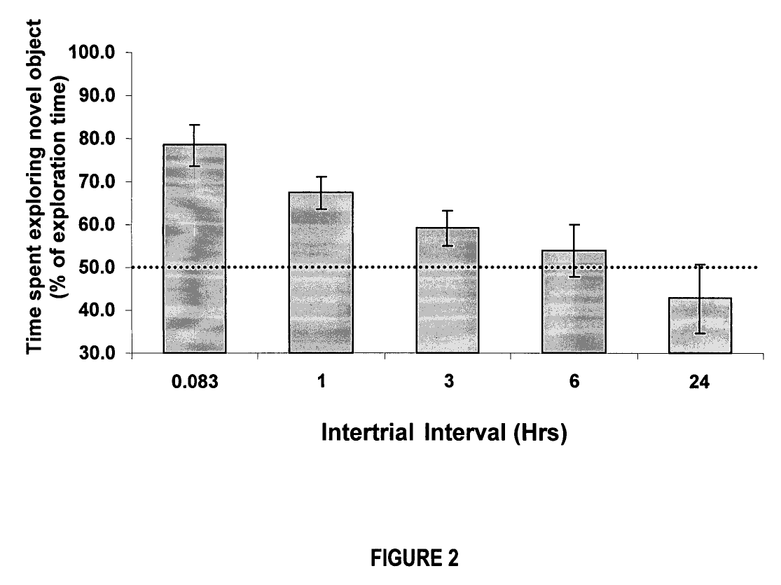

Other exemplary GABA_{B} receptor antagonists useful in the invention include other phosphinic acid analogues of GABA, 2,5 disubstituted-1,4-morpholines, and other compounds. Exemplary antagonists include 3-{1 (S)-[3-(cyclohexylmethyl) hydroxyphosphinyl)-2(S)-hydroxy-propylamino]ethyl}benzoic acid; 3-{1 (R)-[3-(cyclohexylmethyl)hydroxyphosphinyl-2(S)-hydroxy-propylamino]ethyl}benzoic acid; (3-aminopropyl)ethylphosphinic acid (CGP36216); 3-aminopropyl(diethoxymethyl)phosphinic acid (CGP35348); phaclophen (CGP35913); S-(R*,R)]-[3-[[1-(3,4-dichlorophenyl)ethyl]amino]-2-hydroxypropyl](cyclohexylmethyl) phosphinic acid (CGP54626A); 3-[(1R)-1-[[(2S)-2-hydroxy-3-[hydroxyl[(4-methoxyphenyl)methyl]phosphinyl]propyl]amino]ethyl]benzoic acid (CGP62349); CGP54748A, CGP57076A, CGP67588, and CGP80936 (described in Froestl et al., 2003, FIGS. 2, 4 and 7); CGP 46381 [CAS No. 123691-14-5]; CGP 55845 [CAS NO. 149184-22-5]; and CGP-35348 (2-hydroxy-saclofen [CAS No. 123690-79-9]. Other exemplary GABA_{B} receptor antagonists useful in the invention include SCH 50911 [CAS No. 160415-07-6;]; CGP55679; CGP56433; saclofen; and 3-amino-2-hydroxy-N-(4-nitrophenyl)-propanesulphonamide (AHPNS). Other exemplary GABA_{B} receptor antagonists useful in the invention include 2,5 disubstituted-1,4-morpholines and morpholin-2-yl-phosphinic acids (see, e.g, Bolser et al., 1995; Ong et al., 1998). These and other GABA_{B} receptor antagonists are known in the art and/or described in Green et al., 2000; Froestl et al., 2003; Enna, 1997; Bittiger et al., 1993; Olpe et al., 1990; Bolser et al., 1995; Ong et al., 1998; Ong et al., 2001; Kerr et al., 1995; Carai et al., 2004; Pozza et al., 1999; U.S. Pat. Nos. 5,300,679 and 5,064,819; and patent publications U.S. 20020013257; U.S. 20020091250A1; and WO 04000326A1.

Still other exemplary GABA_{B} receptor antagonists useful in the invention include, but are not limited to, propylphosphinic acid derivatives described in U.S. Pat. No. 5,332,729 {e.g., 3-{N-[1 (R)-(3-carboxyphenyl)ethyl]amino}-2(S)-hydroxy-propyl(cyclohex-3-en ylmethyl) phosphinic acid; 3-{N-[1 (S)-(4-carboxyphenyl)ethyl]amino}-2(S)-hydroxy-propyl(cyclohex-3-enylmethyl) phosphinic acid; 3-{N-[1-(4-cyanophenyl)ethyl]amino}-2(S)-hydroxy-propyl(benzyl)phosphinic acid; 3-{N-[1-(3-cyanophenyl)ethyl]amino}-2(S)-hydroxy-propyl(benzyl)phosphinic acid; 3-{N-[1-(4-carboxyphenyl)ethyl]amino}-2(S)-hydroxy-propyl(benzyl)phosphinic acid; 3-{N-[1-(4-carboxyphenyl)ethyl]amino}-2(S)-hydroxy-propyl(benzyl)phosphinic acid; 3-{N-[1-(3-carboxyphenyl)ethyl]amino}-2(S)-hydroxy-propyl(benzyl)phosphinic acid; 3-{N-[1-(3-carboxy-4-methoxyphenyl)ethyl]amino}-2(S)-hydroxy-propyl(benzyl)phosphinic acid; 3-{N-[1-(4-carboxymethylphenyl)ethyl]amino}-2(S)-hydroxy-propyl(cyclohexylmethyl) phosphinic acid; 3-{N-[1-(R)-(3-carboxyphenyl) ethyl]amino}-2-(S)-hydroxy-propyl(diethoxymethyl)phosphinic acid; 3-{N-[1-(S)-(3-carboxyphenyl)ethyl]amino}-2-(S)-hydroxy-propyl(diethoxymethyl)phosphinic acid; 3-{N-[1-(3-cyanophenyl)ethyl]amino}-2(S)-hydroxy-propyl(cyclohexylmethyl)phosphinic acid; 3-{N-[1-(3-carboxyphenyl)ethyl]amino}-2(S)-hydroxy-propyl(cyclohexylmethyl) phosphinic acid; 3-{N-[1-(4-cyanophenyl)ethyl]amino}-2(S)-hydroxy-propyl(cyclohexylmethyl)phosphinic acid; 3-{N-[1 (S)-(4-cyanophenyl)ethyl]amino}-2(S)-hydroxy-propyl(cyclohexylmethyl)phosphinic acid; 3-{N-[1 (R)-(4-cyanophenyl)ethyl]amino}-2(S)-hydroxy-propyl(cyclohexylmethyl) phosphinic acid; 3-{N-[1 (S)-(3-cyanophenyl)ethyl]amino}-2(S)-hydroxy-propyl (cyclohexylmethyl)phosphinic acid; 3-{N-[1(S)-(3-cyanophenyl)ethyl]amino}-2(R)-hydroxy-propyl(cyclohexylmethyl) phosphinic acid; 3-{N-[1 (R)-(3-cyanophenyl)ethyl]amino}-2(S)-hydroxy-propyl(cyclohexylmethyl) phosphinic acid; 3-{N-[1-(4-carboxyphenyl)ethyl]amino}-2(S)-hydroxy-propyl(cyclohexylmethyl) phosphinic acid; 3-{N-[1 (S)-(4-carboxyphenyl)ethyl]amino}-2(S)-hydroxy-propyl(cyclohexylmethyl)phosphinic acid; 3-{N-[1(R)-(4-carboxyphenyl) ethyl]amino}-2(S)-hydroxy-propyl(cyclohexylmethyl)phosphinic acid; 3-{N-[1 (S)-(3-carboxyphenyl)ethyl]amino}-2(S)-hydroxy-propyl(cyclohexylmethyl)phosphinic acid; 3-{N-[1(S)-(3-carboxyphenyl)ethyl]amino}-2(R)-hydroxy-propyl(cyclohexylmethyl)phosphinic acid; and 3-{N-[1 (R)-(3-carboxyphenyl)ethyl]amino}-2(S)-hydroxy-propyl(cyclohexylmethyl) phosphinic acid}.

In some embodiments, the antagonist used has an IC50 of from 1 pM to 1 mM, more often from 1 nM to 100 uM. In one embodiment the antagonist is a small (<1000 D or <500 D) molecule. Although the antagonist can be a natural product it is more often synthetic, such as a synthetic organic compound. Preferably, the antagonist can traverse the blood-brain barrier.

Additional GABA_{B} receptor antagonists can be identified using assays known in the art. For example, in vitro and in vivo models can be used to determine whether a compound functionally blocks GABAB-receptor-mediated cellular responses (Olpe et al., 1990; Froestl et al., 1995; Froestl et al., 2004). For example, recombinant GABA_{B} receptors containing the GB1 and/or GB2 subunits can be expressed in cells, and compounds can be screened against such recombinant receptors for their ability to displace a ligand bound to the receptor, or for their ability to trigger a signaling process.

For example, in one assay antagonism of the effects of the GABA_{B} agonist baclofen is determined. Transverse hippocampal slices of 450-μm thickness are obtained from adult male Sprague-Dawley rats and superfused at 33° C. with gassed artificial cerebrospinal fluid. Drugs are bath applied via syringes connected to the main infusion line. Penicillin-induced epileptic-like discharges were strongly and reversibly depressed by 6 μM baclofen. A compound with receptor antagonist activity (e.g., ABPA) is expected to antagonize the depressant action of 6 μM baclofen. See, e.g., Froestl et al., 1995.

In another exemplary assay, the effect of a compound on GABA release can be determined. Activation of presynaptic GABA_{B} receptors causes an inhibition of neurotransmitter release from both inhibitory and excitatory terminals. A compound with receptor antagonist activity (e.g., ABPA) is expected to reduce the release of GABA in slices of rat cerebral cortex stimulated electrically. See, e.g., Froestl et al., 1995.

In another exemplary assay, the ability of a compound to suppress the late inhibitory postsynaptic potential (IPSP) can be assayed. Postsynaptic GABA_{B} receptors activate a potassium conductance that hyperpolarizes the neuron. In hippocampal slices, stimulation of Schaffer collateral/commissural fibers activates these receptors, producing a late IPSP. A compound with receptor antagonist activity (e.g., ABPA) is expected to suppress the late IPSP in electrically stimulated pyramidal neurons. See, e.g., Froestl et al., 1995.

In another exemplary assay, reversal of the effect of paired-pulse stimulation by a receptor antagonist is assayed. As mentioned above, presynaptic GABA_{B} receptors inhibit neurotransmitter release from both inhibitory and excitatory terminals. These separate populations of presynaptic receptors can be activated by endogenously released GABA; however, the level of activation of each population depends on the pattern of afferent input. As a result, activation of presynaptic GABA_{B} receptors strongly influences the balance of excitatory to inhibitory synaptic input and, hence, the excitability of the postsynaptic neuron. In this regard, paired-pulse stimulation of hippocampal slices causes an increase in the duration of the second field excitatory postsynaptic potential (FEPSP) relative to the first FEPSP, a phenomenon that can be blocked by GABA_{B} receptor antagonists. ABPA, for example, abolished this effect at concentrations of 30 to 300 μM (see Froestl et al., 2004). Using this assay, other antagonists can be identified.

In another exemplary assay, the ability to antagonize GABA_{B} receptors in vivo is tested. In chloral hydrate-anesthetized rats, ABPA administered either by the intravenous, intraperitoneal, or oral route appeared to cross the blood-brain barrier and block GABAB-mediated responses of cortical neurons. When baclofen was administered iontophoretically near spontaneously active cortical neurons, it induced a transient but pronounced firing depression. ABPA partially reduced this depressant effect when given at 10 mg/kg i.v., and it completely reduced the effect when given at 30 mg/kg i.v. See e.g., Froestl et al., 2004. Other antagonists can be identified using this assay.

A number other assays to determine functional effects on GABA_{B} receptors have been described in the literature and can be used to identify compounds that are GABA_{B} receptor antagonists (see, e.g., Ong et al., 1998; U.S. Patent Application U.S. 20020091250A1).

It will be appreciated that, in accordance with the methods of the invention, forms of ABPA or other antagonist can be administered in a variety of forms, including salts, hydrates, co-crystals, enantiomers, and prodrugs of the compounds described above and in the cited references.

Administration in Combination of Donepezil and a GABA_{B} Receptor Antagonist

The invention provides methods for improving cognitive function in a subject by administering a GABA_{B} receptor antagonist, e.g., ABPA, in combination with donepezil. As discussed above, it has now been discovered that when donepezil and a GABA_{B} receptor antagonist, e.g., ABPA, are administered in combination they act synergistically and, moreover, provide therapeutic effect even when administered at doses that would be suboptimal or subtherapeutic when administered individually.

As used herein, administration of a GABA_{B} receptor antagonist and donepezil “in combination” includes simultaneous administration and/or administration at different times, such as sequential administration. Simultaneous administration of drugs encompasses administration as co-formulation or, alternatively, as separate compositions taken within 15 minutes of each other. When the drugs are administered simultaneously, the GABA_{B} receptor antagonist and donepezil may be contained in the same dosage (e.g., a unit dosage form comprising both donepezil and ABPA) or in discrete dosages (e.g., the GABA_{B} receptor antagonist is contained in one dosage form and the acetylcholinesterase inhibitor is contained in another dosage form).

The term “sequential administration” as used herein means that donepezil and the GABA_{B} receptor antagonist are administered with a time separation of more than about 15 minutes, such as more than about one hour, e.g., a time separation of from 1 hour to 12 hours, or longer. In one embodiment, the donepezil and receptor antagonist are administered on the same day. For example, ABPA can be taken in the morning and donepezil in the evening. Either GABA_{B} receptor antagonist or donepezil may be administered first.

Another type of sequential administration is any administration regimen in which the two drugs are administered in the same course of therapy. That is, both drugs are administered to a patient over a period of time to improve the patient's cognitive function. For example, the two drugs might be administered on alternate days.

Dosage Schedules

Dosage schedules of the drugs according to the methods of the invention will vary according to the particular compound or compositions selected, the route of administration, the nature of the condition being treated, the age and condition of the patient, the course or stage of treatment, and will ultimately be at the discretion of the attending physician. It will be understood that the amount of GABA_{B} receptor antagonist and donepezil administered will be amounts effective to effect a desired biological effect (e.g., an amount that blocks, suppresses, or reduces GABA_{B} receptor activity, blocks, suppresses, or reduces acetylcholinesterase activity) such as beneficial results, including clinical results (amounts that in combination result in an improvement in cognitive function). It will be understood that an effective amount can be administered in more than one dose and over a course of treatment.

Donepezil may be administered in combination with a GABA_{B} receptor antagonist at a range of doses, for example, a dosage level up to conventional dosage levels when administered alone. A typical daily dosage of donepezil (ARICEPT®) for treatment of the symptoms of Alzheimer's disease is about 5 to 20 mg, and more often 5 to 10 mg. It has now been discovered the amount of donepezil (ARICEPT) can be reduced when co-administered with ABPA or other GABA_{B} receptor antagonist, while maintaining or improving therapeutic effect. Administration in combination allows the physician to reduce the amount of donepezil (ARICEPT) administered (thereby achieving better tolerability). Further, increased efficacy of a given dose of donepezil (ARICEPT) can occur when administered in combination with a GABA_{B} receptor antagonist. Thus, when administered in combination with a GABA_{B} antagonist such as ABPA the dose is usually less than 20 mg daily, less than 15 mg daily, less than 10 mg daily, or less than 5 daily. In one embodiment, the amount of donepezil (ARICEPT) administered in combination with a GABA_{B} antagonist such as ABPA is less than 4.8 mg daily, less than 4 mg daily, less than 3 mg daily, less than 2 mg daily or less than 1 mg daily. In an embodiment, the subject is administered a daily dose of from 0.5 to 20 mg donepezil. In some embodiments the amount of donepezil (ARICEPT) administered is at least about 0.5 mg/day, e.g., between 0.5 and 5 mg daily, between 0.5 and 4 mg daily, between 0.5 and 3 mg daily, or between 1 mg and 10 mg daily. Administration less frequently than daily is also contemplated.

A GABA_{B} receptor antagonist can be administered in combination with donepezil at a wide range of doses, depending, for example, on the characteristics of the antagonists. A typical daily dosage can range from, for example, about 1 mg to about 5000 mg, 10 mg to about 5000 mg, about 100 mg to about 2000 mg, or about 100 mg to about 500 mg depending on the factors mentioned above. When the antagonist is ABPA, the dosage will typically range from 10 mg to 5000 mg per day, such as from 100 mg to 5000 mg per day; such as from 200 mg to 1800 mg per day, such as from 200 mg to 1000 mg per day. A daily dose can be administered at one time or split (e.g., 1800 mg drug may be administered at 600 mg three times per day). An exemplary dosing regimen involves administering a daily dose of about 100 mg to 200 mg. Administration less frequently than daily is also contemplated, for example, every other day or less frequently. Simultaneous administration of GABA_{B} receptor antagonist and donepezil can optionally be combined with supplemental doses of GABA_{B} receptor antagonist and/or donepezil.

In some embodiments, enough GABA_{B} receptor antagonist is administered so as to allow reduction of the normal dose of acetylcholinesterase inhibitor (e.g., a dose required to effect a degree of cognitive function improvement) by at least 5%, at least 10%, at least about 20%, at least about 30%, at least about 40%, or at least about 50% or more. The reduction may be reflected in terms of amount administered at a given administration and/or amount administered over a given period of time (reduced frequency).

The individual drugs, or coformulation, may be administered according to any schedule and frequency that is therapeutically effective. Most often the drugs or combination are administered up to 4 times per day, more often up to 3 times per day, and most often up to 2 times per day, 1 time per day, or it may be administered less often. A sustained release formulation of a GABA_{B} receptor antagonist (e.g., ABPA) and/or donepezil can be used. The frequency of administration may be adjusted over the course of the treatment, based on the judgment of the administering physician. It will be clear from this disclosure that the GABA_{B} receptor antagonist and donepezil can be administered at different dosing frequencies or intervals. For example, a GABA_{B} receptor antagonist can be administered twice daily and donepezil once daily.

In some embodiments, the GABA_{B} receptor antagonist and donepezil are administered in a predetermined ratio. Without intending to limit the invention, in one embodiment, the amount of GABA_{B} receptor antagonist is greater than that of donepezil (measured w/w). For example, in some embodiments, the ratio by weight of donepezil to the GABA_{B} receptor antagonist is in the range of about 1 to 2000, more often in the range of 1 to 200, and sometimes in the range 1 to 10. Other ratios are contemplated.

Subtherapeutic Doses

Surprisingly, administration of donepezil in combination with ABPA or other antagonist provides benefit even when the amount of each drug administered is an amount that is suboptimal (if administered individually). Surprisingly, the combination provides benefit even when the amount of each drug administered is an amount that, if administered individually, would have little or essentially no therapeutic effect.

Thus, in some embodiments, a subtherapeutic amount of donepezil is administered. “Subtherapeutic amount” refers to an amount that is less than the therapeutic amount, that is, less than the amount normally used when an acetylcholinesterase inhibitor, e.g., donepezil, is administered alone to treat disorders involving cognitive dysfunction. More specifically, a subtherapeutic amount of donepezil is an amount (e.g., a lower dose) that does not result in improved cognition when administered to a subject with a disorder involving cognitive impairment. In one embodiment, the amount of donepezil administered is less than 5 mg per day.

In some embodiments, a “subtherapeutic” amount of the GABA_{B} receptor antagonist is used. A subtherapeutic amount of a GABA_{B} receptor antagonist (i.e. a GABA_{B} receptor antagonist that results in improved cognition when administered to a subject with a disorder involving cognitive impairment) is an amount (e.g., a lower dose) that does not result in improved cognition when administered to such a subject.

In some embodiments, both donepezil and a GABA_{B} receptor antagonist are administered at subtherapeutic amounts.

In some embodiments, a “suboptimal” amount or dose of donepezil and/or GABA_{B} receptor antagonist is administered. The suboptimal amount (or dose) is an amount less than the optimal dose, i.e., less than the amount determined to have optimal or maximum therapeutic effect when administered independently. Usually the optimal dose is a dose approved by the FDA or EMA for administration to treat the condition and/or the dose typically prescribed by physicians.

Administering

It will be appreciated that, as used herein, the terms “administering” or “administration of” a drug to a subject (and grammatical equivalents of this phrase) includes both direct administration, including self-administration, and indirect administration, including the act of prescribing a drug. For example, as used herein, a physician who instructs a patient to self-administer a drug and/or provides a patient with a prescription for a drug is administering the drug to the patient.

In another aspect, the invention provides a method entailing (a) advertising the use of donepezil in combination with a GABA_{B} receptor antagonist and (b) selling donepezil to individuals for use in combination with a GABA_{B} receptor antagonist. In one embodiment, the advertising makes reference to a trademark that identifies the donepezil and the donepezil sold in step (b) is identified by the same trademark. In an embodiment the trademark is ARICEPT®. It will be appreciated that the individuals to whom donepezil is sold include corporate persons (corporations) and the like and “selling donepezil to individuals” includes selling to, for example, a medical facility for distribution to patients.

Compositions

The GABA_{B} receptor antagonist and the donepezil can be administered to a subject via any suitable route or routes. Most often, the drugs are administered orally; however, administration intravenously, subcutaneously, intra-arterially, intramuscularly, intraspinally, rectally, intrathoracically, intraperitoneally, intracentricularly, or transdermally, topically, or by inhalation is also contemplated. They can be administered orally, for example, in the form of tablets, troches, capsules, elixirs, suspensions, syrups, wafers, depot injectable formulations, suppositories, sprays, ointments, cremes, gels, inhalants, dermal patches, implants or the like prepared by art recognized procedures. When a solid carrier is used for administration, the preparation may be tablette, placed in a hard gelatine capsule in powder or pellet form or it may be in the form of a troches of lozenge. If a liquid carrier is used, the preparation may be in the forms of a syrup, emulsion, soft gelatine capsule or sterile injectable liquid such as an aqueous or non-aqueous liquid suspension or solution. Therapeutic formulations can be prepared by methods well known in the art of pharmacy, see, e.g., Goodman et al., 2001; Ansel, et al., 2004; Stoklosa et al., 2001; and Bustamante, et al., 1993.

In one aspect, the invention provides pharmaceutical compositions containing a GABA_{B} receptor antagonist and donepezil. In some embodiments, the two drugs are formulated in a single dosage unit (e.g., combined together in one capsule, tablet, powder, vial, etc.). The unit dose may be in any form (e.g., solid, liquid, aerosol, etc.).

In one embodiment the unit dose contains less than 10 mg donepezil, less than 5 mg donepezil, alternatively less than 4 mg donepezil, less than 3 mg donepezil, less than 2 mg donepezil, or less than 1 mg donepezil. In one embodiment the unit dose contains donepezil and ABPA. In one embodiment the unit dose contains ABPA in a range of from 1 mg to 1000 mg, such as from 50 mg to 600 mg.

Generally a “pharmaceutical composition” contains, in addition to the active drug(s), a pharmaceutically acceptable excipient or carrier. In accordance with the present invention, in addition to donepezil and a GABA_{B} receptor antagonist, solid unit dosage forms of the invention generally include a pharmaceutically acceptable carrier and may contain other agents that serve to enhance and/or complement the effectiveness of the combination, including, for example, additional agents known to be useful for treating cognitive function disorder. As used herein, “pharmaceutically acceptable carrier” refers to a solid or liquid filler, diluent, or encapsulating substance, including for example excipients, fillers, binders, and other components commonly used in pharmaceutical preparations, including, but not limited to, those described below. Methods for formulation of drugs generally are well known in the art, and the descriptions herein are illustrative and not limiting.

Hydrophilic binders suitable for use in the formulations of the invention include copolyvidone (cross-linked polyvinylpyrrolidone), polyvinylpyrrolidone, polyethylene glycol, sucrose, dextrose, corn syrup, polysaccharides (including acacia, guar, and alginates), gelatin, and cellulose derivatives (including HPMC, HPC, and sodium carboxymethylcellulose).

Water-soluble diluents suitable for use in the formulations of the invention include sugars (lactose, sucrose, and dextrose), polysaccharides (dextrates and maltodextrin), polyols (mannitol, xylitol, and sorbitol), and cyclodextrins. Non-water-soluble diluents suitable for use in the formulations of the invention include calcium phosphate, calcium sulfate, starches, modified starches, and microcrystalline cellulose.

Surfactants suitable for use in the formulations of the invention include ionic and non-ionic surfactants or wetting agents such as ethoxylated castor oil, polyglycolyzed glycerides, acetylated monoglycerides, sorbitan fatty acid esters, poloxamers, polyoxyethylene sorbitan fatty acid esters, polyoxyethylene derivatives, nonoglycerides or ethoxylated derivatives thereof, sodium lauryl sulfate, lecithins, alcohols, and phospholipids.

Disintegrants suitable for use in the formulations of the invention include starches, clays, celluloses, alginates, gums, cross-linked polymers (PVP, sodium carboxymethyl-cellulose), sodium starch glycolate, low-substituted hydroxypropyl cellulose, and soy polysaccharides. Preferred disintegrants include a modified cellulose gum such as cross-linked sodium carboxymethylcellulose.

Lubricants and glidants suitable for use in the formulations of the invention include talc, magnesium stearate, calcium stearate, stearic acid, colloidal silicon dioxide, magnesium carbonate, magnesium oxide, calcium silicate, microcrystalline cellulose, starches, mineral oil, waxes, glyceryl behenate, polyethylene glycol, sodium benzoate, sodium acetate, sodium chloride, sodium lauryl sulfate, sodium stearyl fumarate, and hydrogenated vegetable oils. Preferred lubricants include magnesium stearate and talc and combinations thereof.

The preferred range of total mass for the tablet or capsule may be from about 40 mg to 2 g, from about 100 mg to 1000 mg, or from about 300 mg to 750 mg.

In one embodiment, the dosage form is designed to minimize contact between the donepezil and the antagonist. For example, dosage forms of the present invention can be in the form of capsules wherein one active ingredient is compressed into a tablet or in the form of a plurality of microtablets, particles, granules or non-perils, which are then enteric coated. These enteric coated microtablets, particles, granules or non-perils are then placed into a capsule or compressed into a capsule along with a granulation of the other active ingredient.

In addition, the present invention provides unit dosage forms that are sustained release formulations of a combination of receptor and donepezil to allow once a day (or less) oral dosing. In one embodiment, the drugs in the sustained release formulations (also called “modified” or “controlled” release forms) are released over a period of time greater than 6 hours, e.g., greater than 12 hours, after administration. Examples of sustained-release formulations for other drugs that can be modified in accordance with the teachings herein to be useful in the present invention are well known in the art, and are, for example, described in U.S. Pat. Nos. 4,970,075; 6,294,195 and 6,077,533.

The invention provides pharmaceutical kits for the treatment of subjects in need of improved cognition, including a package or container containing donepezil and a GABA_{B} receptor antagonist in discrete dosage forms.

### EXAMPLES

### Example 1

This example shows the effect of administration of ABPA in combination with donepezil on the spatial memory of rats as measured in an 8 hour retention test on a twelve-arm radial maze.

Method

12-arm maze test: Behavioral testing was conducted by an experimenter who was blind to drug treatment. 12 Long-Evans rats trained to use a win-shift strategy were given an information trial. During the information trial, 5 of the 12 arms of the 12 arm maze were blocked so that rats were not able to consume food from those blocked arms but could obtain food from each of the 7 open arms. After this session rats were moved to their home cage and placed back in the animal holding room. 8 hours later (memory test) rats were reintroduced into the maze with all arms open and only the previously blocked arms were baited. Memory for the 7 arms in the information session was demonstrated when the rat visits only the previously blocked arms on the memory test. A retroactive memory error is made when the rat enters an arm that was open on the information trial.

Administration of ABPA and donepezil: A within-subject design was employed to examine drug treatments as a single dose. 12 rats (divided into three groups) were used in the experiment. Sixty minutes prior to the information session, the rats were injected intraperitoneally (IP) with 150 mg/kg ABPA (Saegis Pharmaceuticals, Inc.), 3 mg/kg donepezil, or combination of 150 mg/kg ABPA and 3 mg/kg donepezil. Physiological saline (NaCl) was used as vehicle. Treatment order was counterbalanced among three groups such that each group (N=4) received a different order of vehicle, ABPA, donepezil, and ABPA+donepezil injection across days, for a total of three tests for each treatment in all subjects.

Results

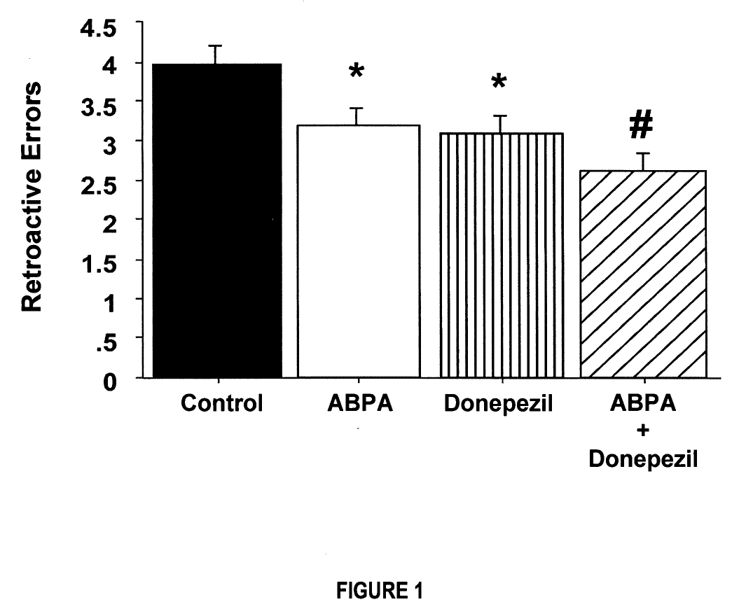

FIG. 1 shows retention test performance on the 12-arm maze after injection of vehicle, ABPA, donepezil, or the combination of the two drugs. ABPA and donepezil each independently improved performance (t(11)=2.69, p<0.02). Performance with combined ABPA and donepezil was significantly improved relative to either ABPA or donepezil alone (p<0.05) and the combined drug treatment differed from vehicle (t(11)=3.82, p<0.003).

### Example 2

This example shows the Object Recognition Task, an animal model used to assess the effects of compounds on memory.

Methods

The object recognition task: The object recognition task is a method to measure a specific form of episodic memory in rats and mice (Ennaceur and Delacour, 1988). It is based on rodents' natural preference for exploring novel objects over familiar objects. The experimental protocol is as follows:

The experiment takes place over a total of 4 days. Objects used for testing included square 60-mL clear glass tablet bottles with a black phenolic cap (“bottle”) or 2½-inch high, 1¼-inch interior diameter aluminum electrical metal tubing conduit couplings (“conduit”). On the first 3 days, the rat was placed into a test box for 15 minutes of habituation. On the fourth day, two identical copies of the same object were arranged in the box, one in each of the near corners about 1/2 inch from the walls—two bottles for half of the rats, two conduits for the other half. The rat was brought to the test room, placed in the middle of the box facing the center of the back wall, and allowed to explore the objects for a 3-minute information trial, after which it was returned to its home cage and to the housing room. After a specified delay, one copy of the original object (“familiar,” not the copy already encountered) and one copy of the other object (“novel”) were arranged in the near corners, with positions counterbalanced to avoid bias, and the rat was placed back in the box for a recognition trial. Behavior during the information and recognition trials was videotaped, and the amount of time spent exploring each object was scored by the same experimenter, who did not know which object was familiar and which novel. The result of scoring is the time spent with the novel object, expressed as a %-age number (the “Recognition Score”)

Normal rats spend more time exploring the novel object, indicating memory for the sample object. Increases in the length of the delay, however, reduce the rat's ability to distinguish between the two objects in the recognition trial. For the testing of cognition-enhancing agents, a delay or inter-trial interval (ITI) is typically chosen at which complete forgetting normally occurs (i.e. where the time spent exploring both the novel and familiar object is equal), as this allows for considerable room for improvement in performance. In a series of pilot experiments where the delay was varied from 5 minutes to 24 hours, the rats' performance decayed to a chance level with an ITI of 6 hours (FIG. 2).

Generation of Dose-Effect Curves for ABPA and Donepezil in the Object Recognition Task

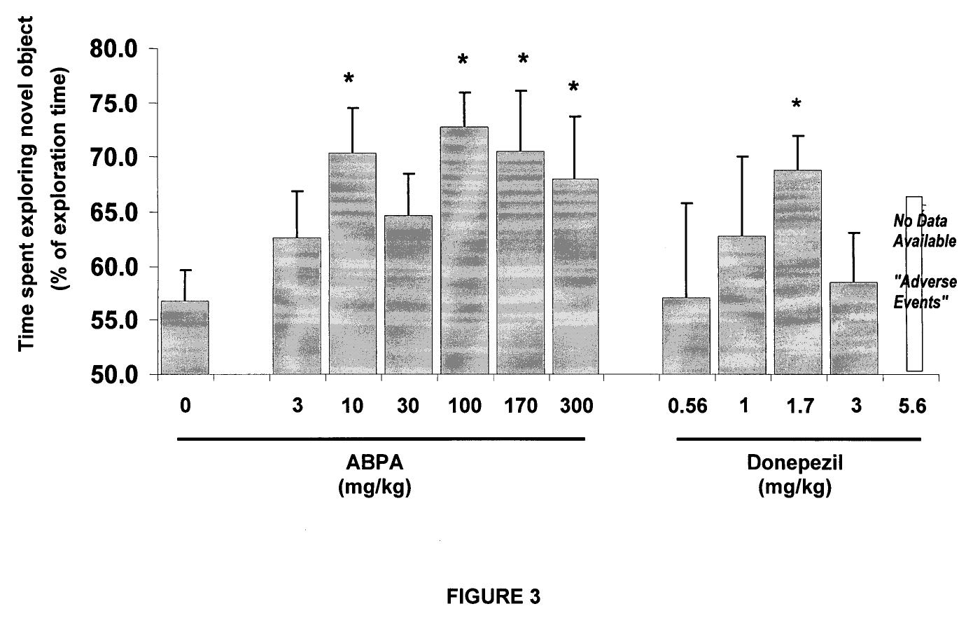

To generate dose-effect curves for donepezil and ABPA, various doses of ABPA and donepezil were administered to rats 30 minutes prior to the information trial and compared to saline-treated controls (FIG. 3). Both drugs were administered by intraperitoneal (IP) injection. When tested at a delay of 6 hours, ABPA significantly enhanced performance when given at a wide variety of doses, i.e., 10, 100, 170, and 300 mg/kg with only 3 and 30 mg/kg showing no beneficial effect.

Donepezil also significantly improved performance in the object recognition task when administered at a dose of 1.7 mg/kg. Administration of doses higher than 1.7 mg/kg began to produce adverse side effects in the rat, a finding that parallels previous studies of AChEIs in general and donepezil in particular.

Combination of Suboptimal Doses of ABPA and Donepezil

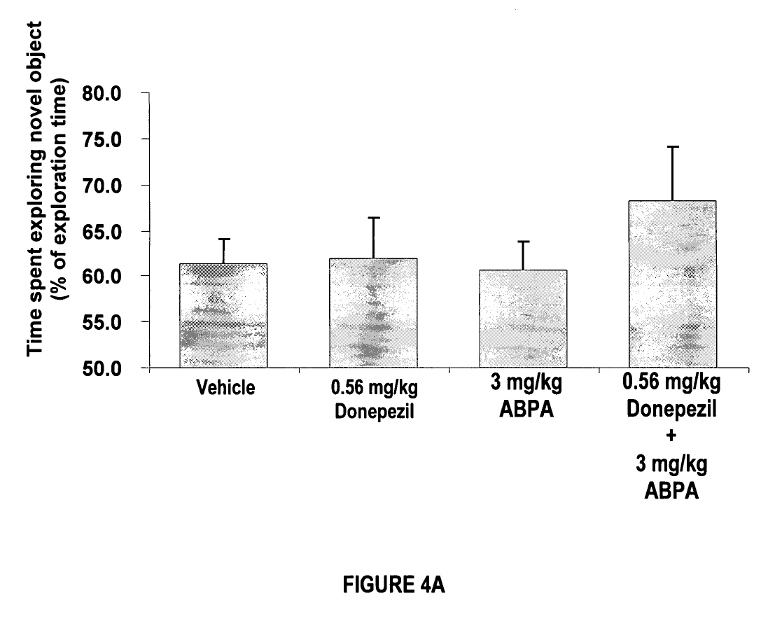

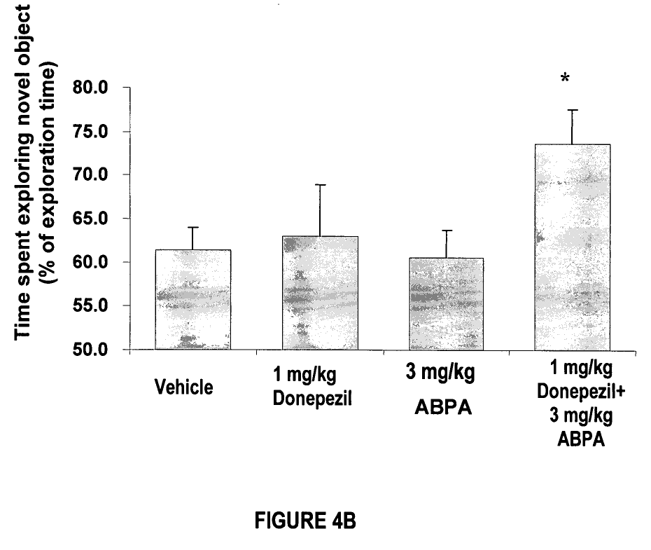

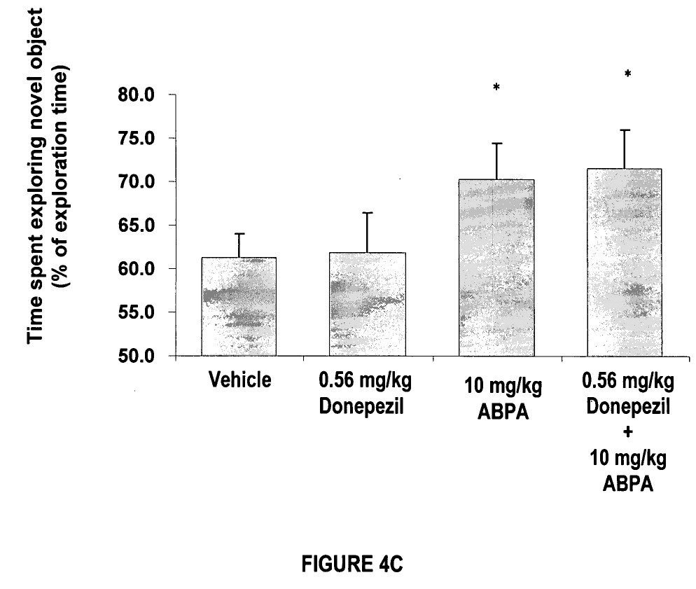

Following the determination of dose-effect curves for both agents, an interaction study was conducted to test whether low doses of ABPA and donepezil in combination have additive or synergistic effects in the object recognition model in rat. Each of two doses of ABPA (3 and 10 mg/kg) were administered either alone or in combination with two different doses of donepezil (0.56 and 1 mg/kg) to rats 30 minutes prior to the information trial. Donepezil alone at 0.56 mg/kg (62% Recognition Score) and 1 mg/kg (63% Recognition Score) and ABPA alone at 3 mg/kg (61% Recognition Score) did not differ from saline (61% Recognition Score). ABPA at 3 mg/kg given with donepezil 0.56 mg/kg tended to improve memory (68% Recognition Score; FIG. 4A), while ABPA at 3 mg/kg given with donepezil 1 mg/kg significantly improved performance in this task (74% Recognition Score; FIG. 4B). Performance of rats treated with this combination (3 mg/kg ABPA and 1 mg/kg donepezil) was significantly better than saline, 3 mg/kg ABPA alone, and 1 mg/kg donepezil alone. This combination also resulted in memory performance slightly better than that produced by the most efficacious doses of either drug in the previous study (100 mg/kg ABPA and 1.7 mg/kg donepezil). The effect of this combination may even be approaching a “ceiling” level, as the best performance seen in this test is 78% Recognition Score, which represents “immediate” recall after a mere 5-minute delay. These data demonstrate an unexpected effect of combining donepezil and ABPA, a synergistic effect attested by the fact that doses ineffective alone are effective when administered in combination.

ABPA alone at 10 mg/kg improved memory (70% Recognition Score), possibly to near the maximal effect, so that any effect of the combinations with donepezil may have been obscured (FIG. 4C).

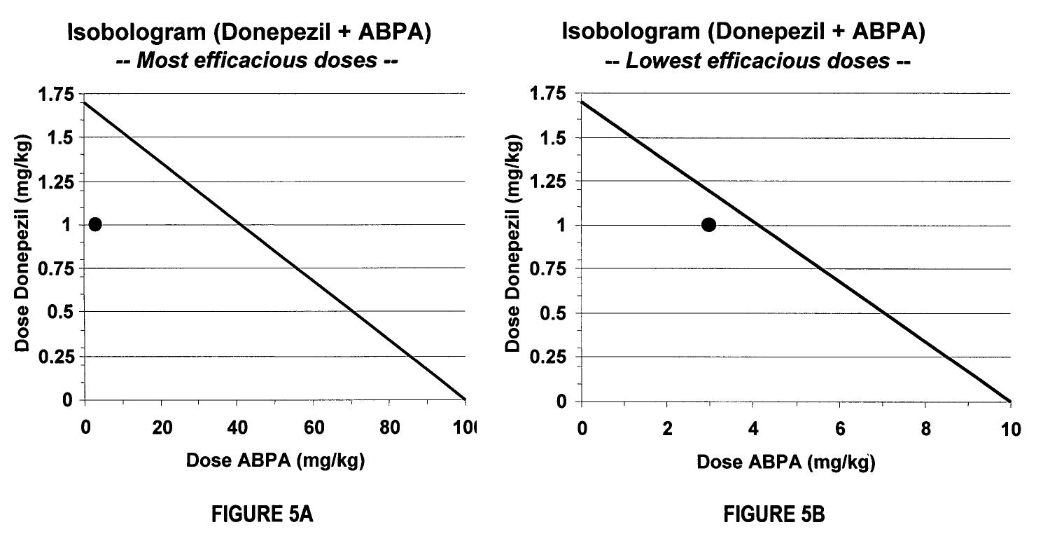

The synergistic effect of the combination of ABPA and donepezil is illustrated by the isobolograms shown in FIG. 5. An isobologram is prepared by plotting equally effective dose pairs (or “isoboles”) for a single effect level (see Tallarida, 2001). From the description of isobolograms from Tallarida (2001), “doses of drug A and Drug B (each alone) that produce a given effect are plotted as axial points in a Cartesian plot. The straight line connecting A and B is the locus of points (dose pairs) that will produce this effect in a simply additive combination. This line of additivity allows a comparison with the actual dose pair that produces this effect level experimentally. It is notable that some dose combinations may be sub-additive (above the line) while others are super-additive or synergistic (below the line).” When the object recognition data described above are plotted as an isobologram, the synergistic effect of 3 mg/kg ABPA and 1 mg/kg donepezil in combination is clearly shown.

### REFERENCES

Ansel et al., 2004, Pharmaceutical Dosage Forms and Drug Delivery Systems 7th Edition, Lippincott Williams & Wilkins Publishers.
Barker et al., 1995, Br. J. Psychiatry 167:642-648.
Barner and Gray, 1998, Ann. Pharmacotherapy 32:70-77.
Bittiger et al., 1993, Trends Pharmacol Sci 14:391-393
Bolser et al., 1995, JPET 274:1393-1448.
Brookmeyer et al., 1998, Am. J. Public Health 88:1337-42.
Bustamante et al., 1993, Physical Pharmacy: Physical Chemical Principles in the Pharmaceutical Sciences 4th Edition.
Carai et al., 2004, Eur J. Pharmacol 504:213-16
Ennaceur and Delacour, 1988, Behavioral Brain Research 31: 47-59.
Enna, 1997, Exp Opin Invest Drugs 6:1319-1325.
Froestl et al., 1995, J. Med. Chem. 38:3313-31
Froestl et al., 2003, II Farmaco 58:173-83.
Froestl et al., 2004, Biochem. Pharmacol. 68:1479-87
Gleiter et al., 1996, J. Clin. Pharmacol., 36:428-38.
Goodman et al., 2001, THE PHARMACOLOGICAL BASIS OF THERAPEUTICS, 10TH EDITION, Mcgraw-Hill Professional.
Green et al., 2000, Brit. J. Pharmacol 131:1766-74
Hanninen et al., 2002, Acta Neurol Scand 106:148-154.
Heese et al., 2000, Neuropharmacology 39:449-62.
Kerr et al., 1995, Eur J Pharmacol. 294:349-52.
Kuriyama et al., 2000, Neurochem Res 25:1233-1239.
Misgeld et al., 1995, Prog Neurobiol 46:423-462.
Mondadori et al., 1993, Behav. Neural Biol. 60: 62-68.
Mondadori et al., 1996, Behav. Brain Res., 77:227-29.
Olpe et al., 1990, Eur. J. Pharmacol. 187:27-38;
Ong and Kerr, 2000, Acta Pharmacol Sin 21:111-123.
Ong et al., 1998, Eur J. Pharmacol. 362:35-41;
Ong et al., 2001, Eur J. Pharmacol 415:191-95
Pittaluga et al., 1997, J. Pharmacol. Exp. Ther., 283:82-90.
Pittaluga et al., 2001, Neuropharmacology 41:301-310
Pozza et al., 1999, Brit. J. Pharm 127:211-19
Rogers and Friedhoff, 1998, Eur. Neuropsych. 8:67-75.
Steulet et al., 1996, Biochem. Pharmacol. 51:613-19.
Stoklosa et al., 2001, PHARMACEUTICAL CALCULATIONS, 11TH EDITION, Lippincott Williams & Wilkins.
Tallarida, 2001, J. Pharm. Exp. Ther. 298:865-872.
Yesavage et al., 2002, J. Psychiatr Res 36:5281-28.
U.S. Pat. No. 5,300,679
U.S. Pat. No. 5,064,819
U.S. Pat. No. 5,332,729
U.S. Pat. No. 4,895,841
U.S. Pat. No. 5,985,864
U.S. Pat. No. 6,140,321
U.S. Pat. No. 6,245,911
U.S. Pat. No. 6,372,760
U.S. Pat. No. 4,970,075
U.S. Pat. No. 6,294,195
U.S. Pat. No. 6,077,533
U.S. Pat. App. U.S. 20020091250A1
U.S. Pat. App. U.S. 20020013257A1
WO 04000326A1

While the present invention has been described with reference to the specific embodiments thereof, it should be understood by those skilled in the art that various changes can be made and equivalents can be substituted without departing from the scope of the invention. In addition, many modifications can be made to adapt a particular situation, material, composition of matter, process, process step or steps, to achieve the benefits provided by the present invention without departing from the scope of the present invention. All such modifications are intended to be within the scope of the claims appended hereto.

All publications and patent documents cited herein are incorporated herein by reference as if each such publication or document was specifically and individually indicated to be incorporated herein by reference. Citation of publications and patent documents is not intended as an indication that any such document is pertinent prior art, nor does it constitute any admission as to the contents or date of the same.

1. A method for improving cognitive function in a subject comprising administering to the subject a GABA_{B} receptor antagonist in combination with donepezil.

2. The method of claim 1, wherein the subject is a human.

3. The method of claim 2, wherein the subject is diagnosed as exhibiting cognitive impairment.

4. The method of claim 1, wherein the GABA_{B} receptor antagonist is 3-aminopropyl-(n-butyl)-phosphinic acid (ABPA).

5. The method of claim 1 wherein the donepezil is donepezil hydrochloride.

6. The method of claim 5, wherein the GABA_{B} receptor antagonist is 3-aminopropyl-(n-butyl)-phosphinic acid (ABPA).

7. The method of claim 4, wherein the subject is administered a daily dose of from 10 mg to 2000 mg ABPA.

8. The method of claim 7, wherein a subtherapeutic amount of donepezil is administered.

9. The method of claim 5, wherein the subject is administered a daily dose of from 0.5 to 20 mg donepezil.

10. The method of claim 9, wherein the subject is administered a daily dose of less than 5 mg donepezil.

11. The method of claim 1, wherein donepezil and the GABA_{B} receptor antagonist are administered simultaneously.

12. The method of claim 10, wherein donepezil and the GABA_{B} receptor antagonist are administered in a single formulation.

13. The method of claim 1, wherein donepezil and the GABA_{B} receptor antagonist are administered sequentially.

14. A pharmaceutical composition comprising donepezil and a GABA_{B} receptor antagonist.

15. The pharmaceutical composition of claim 14 in unit dosage form.

16. The pharmaceutical composition of claim 15, wherein the composition is in a solid form.

17. The pharmaceutical composition of claim 15, wherein the GABA_{B} receptor antagonist is ABPA.

18. The composition of claim 17, wherein the amount of donepezil in the unit dosage is less than about 5 mg.
